SEQUENCE LISTING (3 sequences)

SEQ ID NO: 1 - protein, 214 aa, Artificial Sequence
  The sequence was designed and synthesized
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 224 aa, Artificial Sequence
  The sequence was designed and synthesized
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 20 aa, Artificial Sequence
  The sequence was designed and synthesized
ASPAAPAPASPAAPAPSAPA

(19) United States
(12) Patent Application Publication
(10) Pub. No.: US 2018/0066049 A1
(43) Pub. Date: Mar. 8, 2018

(54) Increasing the half-life of a full-length or a functional fragment of variant anti-human TNF-alpha antibody

(71) Applicant: DNX BIOTECH, LLC, San Diego, CA (US)

(72) Inventors: Rajiv Datar, Irvine, CA (US); Carl K. Edwards, III, San Marcos, CA (US); Scott M. Brown, San Diego, CA (US)

(21) Appl. No.: 15/672,454

(22) Filed: Aug. 9, 2017

Related U.S. Application Data
(63) Continuation of application No. PCT/US2016/016928, filed on Feb. 8, 2016.
(60) Provisional application No. 62/113,894, filed on Feb. 9, 2015.

(51) Int. Cl. C07K 16/24; A61K 38/17

(52) U.S. Cl. CPC C07K 16/241; C07K 2319/31; A61K 38/1774

(57) ABSTRACT

Tumor Necrosis Factor-α (TNFα) promotes an inflammatory response resulting in many clinical problems associated with autoimmune disorders such as rheumatoid arthritis, ankylosing spondylitis, inflammatory bowel disease, psoriasis, hidradenitis suppurativa, and refractory asthma. Dysregulation of TNF production is implicated in a variety of human diseases including Alzheimer's disease, cancer, major depression, and inflammatory bowel disease. These disorders are treated with a TNFα inhibitor. Embodiments herein provide methods of preventing and/or treating acute and chronic inflammation, and autoimmune diseases by administering a prophylactic and/or therapeutic formulation containing an antibody fragment (Fab or F(ab′)₂) of adalimumab modified by conjugation of natural amino acids such as proline, alanine and/or serine (PA/S) by PASylation®, and/or unnatural amino acids such as cysteine and other derivatives, thereby creating a polypeptide possessing none of the processing, preparation, formulation, cost, clinical performance, and other long-term issues of administering PEGylated drugs.

### CROSS-REFERENCE TO RELATED APPLICATIONS

This application claims the benefit of international application serial number PCT/US2016/016928 filed Feb. 8, 2016, which claims the benefit of U.S. provisional application 62/113,894 filed Feb. 9, 2015, each of which is hereby incorporated by reference herein in its entirety.

### FIELD OF THE INVENTION

This invention relates to creation of half-life extended forms of biopharmaceutical molecules for use in the effective, safe, and convenient treatment of immunological, neurological, and cancer diseases, and to half-life modification and drug delivery technologies that increase patient compliance with a course of effective and safe treatment for chronic inflammation and autoimmune diseases such as arthritis, neurological diseases such as Alzheimer's disease, and cancer. Improvements in efficacy, safety, and compliance provide long-term benefits to patients, and reduce costs and clinical burdens.

### BACKGROUND OF THE INVENTION

Monoclonal antibodies are an important class of protein-based medicines. In 2010, at least 25 antibody-based medicines were approved for human therapy and more than 240 antibodies were clinically evaluated (Elbakri, A. et al. (2010) Hum. Immunol. 71 (12), 1243-1250). A small number of antibody fragments (e.g., Fabs) are clinically used. Several classes of engineered antibody fragments (e.g., scFv, diabodies, etc.; Holliger, P. et al. (2005) Nat. Biotechnol. 23 (9), 1126-1136; Nelson, A. L. et al. (2009) Nat. Biotechnol. 27 (4), 1-7; Fischer, N. et al. (2007) Pathobiology 74 (1), 3-14)) and other binding molecules (e.g., scaffolds; Gebauer, M., et al. (2009) Curr. Opin. Chem. Biol. 13 (3), 245-255; Boersma, Y. L. et al. (2011) 22 (6), 849-857; Lipovsek, D. (2011) Protein Eng. 24 (1-2), 3-9) are in development. To capitalize on the therapeutic potential of the antibody fragments and engineered proteins in development, limitations associated with rapid clearance of these proteins from the blood circulation need to be addressed. Currently, a portion of patients suffers from side effects related to immune responses to protein-based medicines (Schellekens, H. (2002) Clin. Ther. 24 (11), 1720-1740; Farrell, R. A. et al. (2012) Rheumatology 51 (4), 590-599; Giezen, T. et al. (2008) J. Am. Med. Assoc. 300 (16), 1887-1896; Singh, S. K. (2011) J. Pharm. Sci. 100 (2), 354-387). Rapid clearance results in the need for more frequent dosing, which increases incidence of side effects and chances for immunogenicity. Efficacy is further compromised by dose-dumping and inability to maintain a therapeutic concentration between doses. The pharmacokinetics of therapeutic proteins is a fundamental property optimized during development to ensure maximal efficacy and safety.

Arthritis and its related manifestations are diseases of global significance affecting more than 53 million adults in the United States and over 1.7 billion people worldwide (American College of Rheumatology, Statistics (2016), www.rheumatology.org/Learning-Center/Statistics; Vos, T. et al., (2012) The Lancet 15; 380(9859):2163-96). The incidence of arthritis will increase substantially in coming decades due to aging populations making the need for better treatments more urgent.

Covalent conjugation of PEG to proteins, Fabs, scFvs, diabodies, and the like is a general approach to extending the half-lives of these life-saving drugs called PEGylation. There are at least 11 PEGylated medicines from a range of different protein classes approved for clinical use (Bailon, P. et al. (2009) Expert Opin. Drug Delivery 6 (1), 1-16; Hamidi, M. et al. (2008) Expert Opin. Drug Discovery 3 (11), 1293-1307; Jevsevar, S. et al. (2010) Biotechnol. J. 5 (1), 113-128; Smith, P. et al. (1985) Anal. Biochem. 150, 76-85; Payne, R. W. et al (2010) Pharm. Dev. Technol., 1-18; Pisal, D. S. et al. (2010) J. Pharm. Sci. 99 (6), 2557-2575; Veronese, F. (2001) Biomaterials 22 (5), 405-417; Veronese, F. M. (2009) Milestones in drug therapy (Parnham, M. J., and Bruinvels, J., Eds.) Birkhauser, Basel; Harris, J. M. et al. (2003) Nat. Rev. Drug Dis. 2 (3), 214-221; Kochendoerfer, G. G. (2005) Curr. Opin. Chem. Biol. 9, 555-560; Pasut, G. et al. (2004) Expert Opin. Ther. Pat. 14 (5), 1-36). While PEGylated medicines are generally clinically safe (Webster, R. et al. (2007) Drug Metab. Dispos. 35 (1), 9-16), additional problems are emerging for PEGylation as a development paradigm for new medicines (European Medicines Agency. (16 Nov. 2012). EMA/CHMP/SWP/647258/2012). Major issues with PEGylation are that it is neither efficient from a processing perspective nor is it site-specific.

The predominant half-life extension technology of PEGylation, developed in the early 1990s, has the following drawbacks: high cost-of-goods; requirement of post-production chemical coupling and processing steps leading to product losses; low biological activity of the drug payload; high viscosity; and accumulation of the drug in organs such as renal tubule cells, macrophages, and choroid plexus epithelial cells, leading to problems with vacuolation (EMEA, November 2012, supra). Clinical development of various PEGylated products such as PEGsunercept®, PEGylated αIL1β Fab, and glycol-PEGylated factor VIIa among others, have been terminated or put on hold.

Many PEGylated medicines used clinically are heterogeneous mixtures produced by nonspecific and inefficient PEG conjugation reactions to different nucleophilic sites on the protein (Bagal et al. (2008) Anal. Chem., 80: 2408-2418). Structurally heterogeneous PEGylated mixtures display different biological properties for each isomer, which is an undesirable characteristic for design of new medicines (Bailor et al. (2009) Expert Opin. Drug Delivery 6 (1), 1-16; Zalipsky (1995) Adv. Drug Delivery Rev. 16, 157-182; Roberts et al. (2002) Adv. Drug Delivery Rev. 54, 459-476). Furthermore, separation and subsequent purification of the heterogeneous mixture for the desired, conjugated single-species moiety results in losses of the product, adding to the high cost-of-goods factor linked to PEGylated biopharmaceutical products.

Tumor necrosis factor-alpha (TNF, TNFα) is a cell signaling protein (cytokine) associated with systemic inflammation. The primary role of TNF is regulation of immune cells. TNF promotes the inflammatory response, which causes many of the clinical problems associated with autoimmune disorders such as rheumatoid arthritis, ankylosing spondylitis, inflammatory bowel disease (IBD), psoriasis, hidradenitis suppurativa, and refractory asthma. In addition, the dysregulation of TNF production is implicated in a variety of human diseases including Alzheimer's disease (Swardfager, W. et al. (2010) Biol Psychiatry 68 (10): 930-941), cancer (Locksley, R. M. et al. (2001) Cell 104 (4): 487-501), major depression (Dowlati, Y. et al. (2010) Biol Psychiatry 67 (5): 446-457) and IBD (Brynskov, J. et al. (2002) Gut 51 (1): 37-43). These disorders are frequently treated with a TNF inhibitor.

A wide array of biological agents to inhibit TNFα have been designed and commercialized (Sedger, L. M. et al. (2014) Cytokine and Growth Factor Reviews 25, 453-472; Schottelius, A. et al. (2004) Experimental Dermatology 13, 193-222). These agents include: a TNFα type II soluble receptor fusion protein (etanercept, Enbrel®, Amgen, Inc.); an anti-human TNFα chimeric (mouse×human) monoclonal antibody (mAb) (infliximab, Remicade®, Centocor Ortho Biotech, Inc); a fully human mAb (adalimumab, Humira®, Abbvie Inc.); a human mAb (golimumab, Simponi®, Centocor Ortho Biotech, Inc.); and a PEGylated Fab fragment anti-TNFα antibody (certolizumab pegol, Cimzia®, UCB Pharma SA). A biosimilar version of inflixamab, CTP-13, i.e., humanized chimeric inflixamab biosimilar IgG₁κ mAB (Rensima®; Celltrion Healthcare Inc.) was approved in South. Korea. Humira® is one of the largest selling drugs in this class. In 2014, global sales of Humira® were estimated at over $13 billion. See, U.S. Pat. No. 6,090,832 for Humira® filed Feb. 9, 1996.

Adalimumab is an IgG antibody composed of two kappa light chains (LCs) each with a molecular weight of approximately 24 kDa and two IgG1 heavy chains (HCs) each with a molecular weight of approximately 49 kDa. See, U.S. Pat. No. 6,090,832. The antibody consists of 1,330 amino acids and has a molecular weight of approximately 148 kDa. Each LC consists of 214 amino acid residues and each HC consists of 451 amino acid residues. See, U.S. Pat. No. 6,090,832.

The active ingredient was produced by cell culture using Chinese Hamster Ovary (CHO) cells and was tested for viral clearance in a previous study (European Medicines Agency. Assessment Report on Humira (2004) WC500050867). Limited clearance values for small non-enveloped virus such as minute virus of mice (MVM) result from the purification process; therefore, each harvest was tested for the presence of viruses and a specific assay (Q-PCR) was used to detect MVM. Adalimumab was administered to adult patients with rheumatoid arthritis (RA) as a 40 mg subcutaneous (s.c.) injection every other week (eow). Human antibodies against adalimumab (AAA) were observed to occur in up to 12.4% of the patients in clinical trials, and because of this efficacy failure, dosage was increased from 40 mg eow, to 40 mg every week. Concomitant use with methotrexate (MTX) reduced the clearance of adalimumab and the likelihood of AAA formation (EMEA 2004, supra).

Certolizumab pegol (Cimzia®) is a recombinant, humanized antibody Fab fragment specific for human TNFα (European Medicines Agency. Assessment Report on Cimzia (2009), WC500069735). The drug is a chimeric mAb/Fab fragment, composed of a murine CDR specifically directed against human TNF-α grafted into a constant folate receptor (FR) of a human κ-LC and IgG4 Fab. The LC contains 214 amino acid residues, and the HC contains 229 amino acid residues. The molecular mass of the Fab′ antibody fragment is 47.8 kDa. The Fab fragment is manufactured in E. coli, purified, and subsequently conjugated through a maleimide group to 40 kDa PEG to extend the plasma half-life to that of the whole antibody, which generally has a total molecular weight of approximately 91 kDa. The Fab fragment administered to adult patients with RA at 400 mg s.c. at weeks 0, 2 and 4, followed by a maintenance dose of 200 mg every 2 weeks, resulted in occurrence of antibodies at an approximately three-fold increase in certolizumab pegol clearance. Higher clearance in antibody positive subjects resulted in reduced clinical efficacy of the drug. Furthermore, pharmacokinetic data of certolizumab pegol indicates that the drug undergoes proteolysis and excretion in urine due to the protein characteristics of the Fab fragment. The PEG component appears in tissues, including liver, spleen, kidneys, heart, lungs, brain, and mesenteric lymph nodes. Data from SDS-PAGE analyses show only the 40 kDa material (PEG) in the urine of rats. Thus, the Fab catabolizes prior to excretion of the two 20 kDa PEG chains linked by a lysine residue. However, the metabolic fate of the maleimide linker was not determined from these data. The characteristic of acting as a ‘hot-spot’ for immunological reactions is a well-known feature of linker technology (EMEA 2009, supra).

As many as 40% of patients with established RA fail to respond adequately to non-biologic DMARDs and about 60% of patients fail to achieve a major clinical American College of Rheumatology (ACR) response despite early disease-modifying anti-rheumatic drug (DMARD) and/or biological therapy. Even among responders, the majority do not achieve remission (EMEA 2009, supra). Therefore, there is a need for new biological therapies and mechanisms for the treatment of RA.

### SUMMARY OF THE INVENTION

Various embodiments of the invention herein provide a composition for preventing or treating a subject for at least one of an inflammation, an autoimmune disease, a neurological disease, and a cancer, the composition including: a full-length antibody or a functional antibody fragment that is an anti-human TNFα antibody; and an adduct covalently linked to the full-length antibody or the functional antibody fragment that increases half-life of the composition in the subject, and the composition having decreased immunogenicity than the full-length antibody or the functional antibody fragment alone, or than a corresponding PEGylated form of the full-length antibody or the functional antibody fragment.

Certain embodiments of the composition provide the full-length antibody or the functional antibody fragment, which is at least one antibody class of proteins selected from the group consisting of: IgG, IgM, IgA, IgD, and IgE. Certain embodiments of the composition provide the full-length antibody or the functional antibody fragment as from the IgG class of proteins. An aspect of the composition provides the functional antibody fragment as a Fab or a F(ab′)₂. An aspect of the composition provides the full-length antibody or the functional antibody fragment as adalimumab. Certain embodiments of the composition provide the full-length antibody or the functional antibody fragment as human or humanized. Certain embodiments of the composition herein provide the amino acid sequence of the full-length antibody or the functional antibody fragment includes at least a portion of a human antibody. An aspect of the composition provides the composition as biodegradable in vivo in the subject.

Certain embodiments of the composition herein provide the Fab or the F(ab′)₂ as a recombinant mutagenized protein. Certain embodiments of the composition herein provide the Fab of the F(ab′)₂ as a proteolytic product of a digest of the full-length antibody. Certain embodiments of the composition herein provide the Fab or the F(ab′)₂ as encoded by a nucleic acid obtained by at least one technique selected from chemical synthesis, cDNA, genomic library screening, expression library screening, or polymerase chain reaction (PCR). An aspect provides the composition as biodegradable by kidney enzymes of the subject. Certain embodiments of the composition herein provide the adduct as a polypeptide containing proline and alanine. Certain embodiments of the composition herein provide the adduct as a polypeptide that further includes serine (PAS polypeptide). Certain embodiments of the composition herein provide the adduct includes naturally occurring sugars. For example, the sugars include glucuronic acid and the N-acetylglucosamine. More specifically, the sugars include heparosan.

Certain embodiments of the composition herein provide the adduct includes a linear polypeptide containing natural amino acid residues. Certain embodiments of the composition herein provide the adduct includes a linear polypeptide containing unnatural amino acid residues. Certain embodiments of the composition herein provide the adduct includes a nonlinear polypeptide. Certain embodiments of the composition herein provide the adduct increases the half-life of the full-length antibody or the functional antibody fragment at least about 10-fold. Certain embodiments of the composition herein provide the adduct increases the half-life of the full-length antibody or the functional antibody fragment by a factor of at least about 300-fold. Certain embodiments of the composition herein provide the PAS polypeptide form a monodisperse mixture. Certain embodiments of the composition herein provide the adduct as covalently linked at the C terminus of the full-length antibody or the functional antibody fragment or the N terminus of the full-length antibody or the functional antibody fragment. Certain embodiments of the composition herein provide the adduct as a plurality of adducts, and a first adduct is covalently linked at the N terminus and a second adduct is covalently linked at the C terminus of the full-length antibody or the functional antibody fragment.

Certain embodiments of the composition herein provides the adduct is covalently linked to the full-length antibody or the functional antibody fragment at a position internal to the N terminus or the C terminus. Certain embodiments of the composition herein provide the adduct as a plurality of adducts, and each of the plurality as covalently linked to one of a plurality of positions on the full-length antibody or the functional antibody fragment. Certain embodiments of the composition herein provides the adduct includes at least one drugs selected from: an anti-inflammatory drug, a steroidal drug, a non-steroidal drug, or an immunotoxin. For example, the anti-inflammatory drug is methotrexate. Certain embodiments of the composition herein provide the adduct as located at or in close proximity of an immunogenic site of the full-length antibody or the functional antibody fragment and masks immunogenicity. Certain embodiments of the composition herein provide the adduct as at least about 200 amino acid residues. For example, the adduct includes at least about 1200 amino acid residues.

Certain embodiments of the composition herein provide the covalent linkage includes two adducts, each having a length of at least about 200 amino acid residues. Certain embodiments of the composition herein provides the half-life of the composition in vivo as at least about 25 hours, at least about 75 hours, at least about 125 hours, at least about 175 hours, at least about 225 hours, or at least about 275 hours. Certain embodiments of the composition herein provide the composition further includes an affinity tag for chromatographic purification. Certain embodiments of the composition herein provide the composition accumulates at an inflamed site or in diseased cells to treat the subject. Certain embodiments of the composition herein provide the adduct forms a random coil conformation domain (RCCD).

Various embodiments of the invention herein provide a method of preventing or treating a subject for at least one of an inflammation, an autoimmune disease, a neurological disease, and a cancer, the method including: engineering a composition including a full-length antibody or a functional antibody fragment that is a Fab or a F(ab′)₂ covalently bound to an adduct, the composition increasing the half-life of the composition in the subject, and the composition containing the adduct as less immunogenic than that full-length antibody or the functional antibody fragment which is PEGylated; and administering the composition to the subject.

Certain embodiments of the method herein provide the method further includes prior to administering, formulating the composition in a form that is effective for a prophylactic use. Alternatively, the method further includes prior to administering, formulating the composition in a form that is effective for a therapeutic use. Certain embodiments of the method herein provide the engineering step includes covalently binding an adalimumab to the adduct. Certain embodiments provide the method further includes prior to administering, genetically conjugating the adduct to the full-length antibody or the functional antibody fragment. Certain embodiments provide the method further includes prior to administering, chemically conjugating the adduct to the full-length antibody or the functional antibody fragment. Certain embodiments provide the method further includes prior to administering, increasing the half-life of the full-length antibody or the functional antibody fragment by conjugating a PAS polypeptide or naturally occurring sugar molecules including heparosan, to the full-length antibody or the functional antibody fragment.

Certain embodiments provide the method further includes prior to administering, expressing the composition in prokaryotic cells. Certain embodiments provide the method further includes prior to administering, expressing the composition in eukaryotic cells. Certain embodiments provide the engineering step further includes covalently conjugating the full-length antibody or the functional antibody fragment to a PAS polypeptide. Certain embodiments provide the method further includes prior to administering, forming the Fab or the F(ab′)2 using mutagenesis. Certain embodiments provide the method further includes prior to administering, digesting the full-length antibody to form the Fab or the F(ab′)₂.

### BRIEF DESCRIPTION OF THE DRAWINGS

FIG. 1 is a diagram of the problem and the optimal solutions provided herein to half-life extension and effective delivery of biopharmaceutical drugs.

FIG. 2 is a graph of de-convoluted zero-charge mass spectra showing highly polydisperse nature of PEG residues in prior art compositions used for increasing the half-lives of biopharmaceutical compositions. See, Bagal et al., Anal. Chem., 80: 2408-2418 (2008).

FIG. 3 is a drawing of the structure formed from natural amino acids or from a combination of natural and unnatural amino acids having length “n” in a linear polypeptide polymer.

FIG. 4 is a graph of mass spectroscopy data indicating the size distribution and monodisperse nature of the polypeptide structure of FIG. 3.

FIG. 5 is a graph that compares the viscosities among polypeptides and PEG polymers having various lengths of amino acid residues exemplified by the repeating structure of the PAS polypeptide in FIG. 3 and PEG polymers in the preferred molecular weight range.

FIG. 6A is a ribbon-model based on x-ray crystallography data of the three-dimensional structure of a full-length adalimumab antibody.

FIG. 6B is an illustration of the full-length adalimumab antibody with hypervariable regions labeled. “Grafted” as used in FIG. 6B refers to genetically altered amino acid sequence that is recombinantly expressed to humanize the antibody.

FIG. 7 is an illustration showing steps in the process of synthesis of antibody fragments (Fab) from a full-length antibody.

FIG. 8 is an illustration of the process of synthesis of biopharmaceutical molecules with an extended half-life. The molecules contain components of used in the process of FIG. 7 and variants thereof and PAS polypeptides, 10, and variants thereof shown in FIG. 3.

FIG. 9 is an illustration of the increase in the hydrodynamic volume of Fab conjugated to different variants of the PAS polypeptide.

FIG. 10 is a plot of elimination half-life of biopharmaceutical molecules as a function of body weight using the principles of interspecies allometric scaling for the biopharmaceutical molecules produced by the process in FIG. 8 and variants thereof across several clinically-relevant species.

FIG. 11A and FIG. 11B are amino acid sequences of the Fab of adalimumab light (SEQ ID NO: 1) and heavy chains (SEQ ID NO: 2), respectively.

FIG. 12A is a plasmid map and the restriction sites used for genetically fusing a PAS polypeptide to a fragment of adalimumab.

FIG. 12B is a plasmid map and the restriction sites used for genetically fusing a PAS polypeptide and a His6 tag to a fragment of adalimumab.

The sequence listing material in computer readable form ASCII text file (5 kilobytes) created Aug. 2, 2017 entitled “12998-010_SequenceListing_08092017”, containing sequence listing numbers 1-3, has been electronically filed herewith and is incorporated by reference herein in its entirety.

### DETAILED DESCRIPTION OF THE INVENTION

Half-life extension technologies have been developed such as the polypeptide-based, random-coil domain (RCD) technology called PASylation® (Payne et al. (2010) Pharm. Dev. Technol., 1-18; Pisal et al. (2010) J. Pharm. Sci. 99 (6), 2557-2575; Veronese. (2001) Biomaterials 22 (5), 405-417; Veronese (2009) Milestones in drug therapy (Parnham, M. J., and Bruinvels, J., Eds.) Birkhauser, Basel). See, Skerra et al., WO 2011/144756 published Nov. 24, 2011 and Skerra et al., WO 2008/155134 published Dec. 24, 2011, each of which is hereby incorporated by reference in its entirety The polypeptides in PASylation® contain sequences of amino acids proline, alanine, and optionally serine (PA/S or PAS) residues. The polymer, which is a combination of amino acid residues, results in cancellation of the distinct secondary structure preferences of each amino acid residue to form a stably disordered polypeptide.

Issues of immunogenicity, clearance, viscosity, routes, methods, and frequency of administration affect production and use of products currently on the market. In the 21^{St} century, half-life extension technologies such as the polypeptide-based PASylation® technology have emerged to modify molecules such as Fabs, scFVs, and diabodies, in a manner that circumvents poor performance issues of PEGylation (Schlapschy et al. (2013) Protein. Engineering, Design & Selection 26:8 489-501; Morath et al. (2015) pubs.acs.org/molecularpharmaceutics; Skerra et al., WO2008155134A1 published Dec. 24, 2008; Skerra et al., WO2011144756A1 published Nov. 24, 2011). Attachment of biologically active proteins to at least one PAS polypeptide, which contains a domain with an amino acid sequence that assumes a random coil conformation, has been observed to increase stability in vivo and/or in vitro compared to the protein in its native state lacking this adduct.

PASylation® provides advantages that PEGylation cannot, for example: high target affinity maintenance; lower elicitation immunogenicity in preclinical trials due to use of natural linkers; efficient biodegradation by kidney enzymes, with stability in the blood stream; absence of polydispersity; and no requirement for in vitro coupling steps, thereby reducing the cost of goods. PASylated molecules have lower viscosity than PEG of the comparable molecular weight, and the half-life of these molecules is tunable from about 10-fold to about 300-fold increase. These advantages render the PAS polypeptide more efficacious, safer, and more convenient because of lowered dosing and frequency of administration than PEGylated or non-altered proteins resulting in an increase in patient compliance.

Use of existing drugs to treat immunosuppressed patients has serious drawbacks. These drugs are not modified to be rapidly cleared from the body, requiring compensatory higher quantities and/or by more frequent dosing regimens than is desirable.

PASylated proteins provided herein mask the immuno-suppressive nature of the biopharmaceutical drug and simultaneously increase its half-life in the body. Consequently, the drug is not rejected by the body, does not result in immune reactions, and is dosed at lower quantities or frequency.

Modification of one or more types of the antibody fragments of adalimumab was observed to improve the therapeutic outcomes for patients suffering from life-long diseases such as arthritis and other immunology-based inflammation and autoimmune diseases. Adalimumab is a full-length immunoglobulin (IgG1) molecule with optimized HCs and LCs. For production by mammalian cell technology, a CHO host cell is transfected with a plasmid vector containing the expression cassettes for adalimumab HCs and LCs. Use of recombinantly produced antibody fragments allows the production to be carried out in a prokaryotic host cell such as E. coli, which is considerably easier from the perspective of large-scale production of biological products, compared with a mammalian cell technology counterpart.

An embodiment of the compositions and methods herein provides a functionally active, truncated form of adalimumab, modified for increased half-life, which is produced in its modified form using a molecular biology approach, instead of using post-production chemical coupling methods and technologies as is used for PEG.

An embodiment herein provides methods of preventing and/or treating acute and chronic inflammation and autoimmune diseases by administering a prophylactic and/or therapeutic formulation containing of one or more types of recombinant antibody fragments (Fab) of adalimumab (Fab_{adal}), which are conjugated to a polypeptide containing natural or unnatural amino acids and having specific length “n”. The modification was observed to result in longer and more effective treatment because unmodified Fabs are cleared quickly from the human body.

Techniques for replacing, inserting, or deleting one or more selected amino acid residues, such as mutagenesis are well known to one of ordinary skill in the art. See e.g., U.S. Pat. No. 4,518,584, which is hereby incorporated by reference in its entirety. Principal variables in the construction of each amino acid sequence variant are the location of the mutation site and the nature of the mutation. The location of each mutation site and the nature of the mutation will depend on the biochemical characteristic(s) to be modified. Each mutation site is modified achieved individually or in series by: substituting first with conservative amino acid choices, depending on results substituting radical selections, deleting the target amino acid residue, or inserting amino acid residues adjacent to the located. These techniques make combinations of deletions, insertions, and substitutions in the amino acid sequence of adalimumab and its fragments to create a variety of truncated forms that are biologically active.

In addition to mutagenesis techniques described above, the process of antibody digestion to create Fabs from whole antibodies is also well-known in the art. Proteolytic digestion of a full-length antibody with papain yields Fab and digestion with IdeS enzyme (FabRICATOR, Genovis, Sweden) yields F(ab′)2 as described herein.

Fabs have been chemically modified in vitro with any of water-soluble, non-biological, or synthetic polymers, to create a multitude of chemically-derivatized Fab structures. See, U.S. Pat. No. 6,989,147, which is hereby incorporated by reference in its entirety. PEG is the best known of these synthetic, non-biodegradable polymers. U.S. Pat. No. 6,989,147 shows that the average molecular weight of the PEG polymer is preferably between about 5 kDa and about 50 kDa, more preferably between about 12 kDa and about 40 kDa, and most preferably between about 20 kDa and about 35 kDa.

Generally, the higher the molecular weight of the PEG and/or the more branches of the PEG polymer coupled to the protein of interest, the higher the polymer:protein ratio. A higher the polymer:protein ratio results in a higher viscosity of the chemically-coupled product, which negatively affects ease-of-injection and mode-of-delivery factors. Proteins chemically conjugated to PEG polymers in the molecular weight range of 20 kDa to 35 kDa have viscosities of up to 400 cP. See, U.S. Pat. No. 7,700,722. At these high viscosities, injection times are long (about 80 seconds or more), or alternatively, significantly thicker gauge needles are used (e.g. 23 G) resulting in extremely painful injections.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, 22^{nd} Ed.; Gennaro, Mack Publishing, Easton, Pa. (2012), which is hereby incorporated by reference in its entirety, provides various carriers used in formulating pharmaceutical compositions and known techniques for the preparation thereof. Examples of materials which serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants may also be present in the composition, the choice of agents and non-irritating concentrations to be determined according to the judgment of the formulator.

### Preparation of Antibodies

An antibody or antibody fragment used herein in compositions and methods recognizes and binds preferentially to TNFα. The anti-TNFα antibody employed is a monoclonal antibody or a polyclonal antibody and may be obtained by immunizing an appropriate animal through a known technique. Alternatively, a commercial available antibody is used in the compositions and methods herein. These antibodies are used alone or in appropriate combination. Alternatively, there is an immunological assay method is employed (WO 2005/038458), which method includes reacting TNFα contained in a biological sample with one or more members selected from a reducing agent, an acid or a salt thereof, a surfactant, and a protease other than chymotrypsin to convert TNFα multimers to a certain specific form; and targeting the converted product.

### Therapeutically Effective Dose

Compositions, according to the method of the current invention, are administered using any amount and by any route of administration effective for preventing or treating a subject for an inflammation or an autoimmune disease. An effective amount refers to a sufficient amount of the composition to beneficially prevent or ameliorate the symptoms of the disease or condition.

The exact dosage is chosen by the individual physician in view of the patient to be treated. Dosage and administration are adjusted to provide sufficient levels of the active agent(s) or to maintain the desired effect in a subject. Additional factors which may be taken into account include the severity of the disease state, e.g., liver function, cancer progression, and/or intermediate or advanced stage of macular degeneration; age; weight; gender; diet, time; frequency of administration; route of administration; drug combinations; reaction sensitivities; level of immunosuppression; and tolerance/response to therapy. Long acting pharmaceutical compositions are administered, for example, hourly, twice hourly, every three to four hours, daily, twice daily, every three to four days, every week, or once every two weeks depending on half-life and clearance rate of the particular composition.

The active agents of the pharmaceutical compositions of embodiments of the invention are preferably formulated in dosage unit form for ease of administration and uniformity of dosage. The expression “dosage unit form” as used herein refers to a physically discrete unit of active agent appropriate for the patient to be treated. The total daily usage of the compositions of the present invention will be decided by the attending physician within the scope of sound medical judgment. For any active agent, the therapeutically effective dose is estimated initially either in cell culture assays or in animal models, potentially mice, pigs, goats, rabbits, sheep, primates, monkeys, dogs, camels, or high value animals. The cell-based, animal, and in vivo models provided herein are also used to achieve a desirable concentration, total dosing range, and route of administration. Such information is used to determine useful doses and routes for administration in humans.

A therapeutically effective dose refers to that amount of active agent that ameliorates the symptoms or condition or prevents progression of the disease or condition. Therapeutic efficacy and toxicity of active agents are determined by standard pharmaceutical procedures in cell cultures or experimental animals, e.g., ED₅₀ (dose therapeutically effective in 50% of the population) and LD₅₀ (dose lethal to 50% of the population). The dose ratio of toxic to therapeutic effects is the therapeutic index, which is expressed as the ratio, LD₅₀/ED₅₀. Pharmaceutical compositions having large therapeutic indices are preferred. The data obtained from cell culture assays and animal studies are used in formulating a range of dosage for human use.

### Administration of Pharmaceutical Compositions

As formulated with an appropriate pharmaceutically acceptable carrier in a desired dosage, the pharmaceutical composition or methods provided herein is administered to humans and other mammals for example topically for skin tumors (such as by powders, ointments, creams, or drops), orally, rectally, mucosally, sublingually, parenterally, intracisternally, intravaginally, intraperitoneally, intravenously, subcutaneously, bucally, sublingually, ocularly, or intranasally, depending on preventive or therapeutic objectives and the severity and nature of the cancer-related disorder or condition.

Injections of the pharmaceutical composition include intravenous, subcutaneous, intra-muscular, intraperitoneal, or intra-ocular injection into the inflamed or diseased area directly, for example, for esophageal, breast, brain, head and neck, and prostate inflammation.

Liquid dosage forms are, for example, but not limited to, intravenous, ocular, mucosal, pharmaceutically acceptable emulsions, microemulsions, solutions, suspensions, syrups, and elixirs. In addition to at least one active agent, the liquid dosage forms potentially contain inert diluents commonly used in the art such as, for example, water or other solvents; solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols, fatty acid esters of sorbitan, and mixtures thereof. Besides inert diluents, the ocular, oral, or other systemically-delivered compositions also include adjuvants such as wetting agents, emulsifying agents, and suspending agents.

Dosage forms for topical or transdermal administration of the pharmaceutical composition herein include ointments, pastes, creams, lotions, gels, powders, solutions, sprays, inhalants, or patches. The active agent is admixed under sterile conditions with a pharmaceutically acceptable carrier. Preservatives or buffers may be required. For example, ocular or cutaneous routes of administration are achieved with aqueous drops, a mist, an emulsion, or a cream. Administration is in a therapeutic or prophylactic form. Certain embodiments of the invention herein contain implantation devices, surgical devices, or products which contain disclosed compositions (e.g., gauze bandages or strips), and methods of making or using such devices or products. These devices may be coated with, impregnated with, bonded to or otherwise treated with the composition herein.

Transdermal patches have the added advantage of providing controlled delivery of the active ingredients to the eye and body. Such dosage forms can be made by dissolving or dispensing the compound in the proper medium. Absorption enhancers are used to increase the flux of the compound across the skin. Rate is controlled by either providing a rate controlling membrane or by dispersing the compound in a polymer matrix or gel.

Injectable preparations of the pharmaceutical composition, for example, sterile injectable aqueous or oleaginous suspensions are formulated according to the known art using suitable dispersing agents, wetting agents, and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or a suspending medium. For this purpose, bland fixed oil including synthetic mono-glycerides or di-glycerides is used. In addition, fatty acids such as oleic acid are used in the preparation of injectables. The injectable formulations are sterilized prior to use, for example, by filtration through a bacterial-retaining filter, by irradiation, or by incorporating sterilizing agents in the form of sterile solid compositions, which are dissolved or dispersed in sterile water or other sterile injectable medium. Slowing absorption of the agent from subcutaneous or intratumoral injection was observed to prolong the effect of an active agent. Delayed absorption of a parenterally administered active agent is accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release is controlled. Examples of other biodegradable polymers include poly (orthoesters) and poly(anhydrides). Depot injectable formulations are also prepared by entrapping the agent in liposomes or microemulsions that are compatible with body tissues.

Solid dosage forms for oral administration include capsules, tablets, pills, powders, and granules. In solid dosage forms, the active agent is mixed with at least one inert, pharmaceutically acceptable excipient or carrier such as sodium citrate, dicalcium phosphate, fillers, and/or extenders such as starches, sucrose, glucose, mannitol, and silicic acid; binders such as carboxymethylcellulose, alginates, gelatin, polyvinylpyrrolidinone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents, for example, cetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using excipients such as milk sugar as well as high molecular weight PEG and the like. The solid dosage forms of tablets, dragees, capsules, pills, and granules are prepared with coatings and shells such as enteric coatings, release controlling coatings, and other coatings known in the art of pharmaceutical formulating. In these solid dosage forms, the active agent(s) are admixed with at least one inert diluent such as sucrose or starch. Such dosage forms also include, as is standard practice, additional substances other than inert diluents, e.g., tableting lubricants and other tableting aids such as magnesium stearate and microcrystalline cellulose. In the case of capsules, tablets and pills, the dosage forms may also include buffering agents. The composition optionally contains opacifying agents that release the active agent(s) only, preferably in a certain part of the intestinal tract, and optionally in a delayed manner. Examples of embedding compositions include polymeric substances and waxes.

### Recombinant Expression and Preparation of Fusion Polynucleotides

Nucleic acid sequences encoding the light and/or heavy chains of adalimumab are readily obtainable in a variety of ways, for example, chemical synthesis, cDNA or genomic library screening, expression library screening, and/or polymerase chain reaction (PCR) amplification of cDNA. These methods and others, which are used for isolating such nucleic acid sequences, are set forth in Sambrook et al. Molecular Cloning: A Laboratory Manual. Cold Spring Harbor Laboratory Press, Cold Spring Harbor, N.Y. 1989, Ausubel et al., eds. Current Protocols in Molecular Biology. Current Protocols Press. 1994, and Berger et al. Methods in Enzymology: Guide to Molecular Cloning Techniques. Vol. 152, Academic Press, Inc., San Diego, Calif. 1987, each of which is incorporated by reference in its entirety.

Chemical synthesis of nucleic acid sequences, which encode fragments of adalimumab, is accomplished using methods well-known in the art, such as those set forth by Engels et al. (1989) Angew. Chem. Intl. Ed. 28:716-734 and Wells et al. (1985) Gene. 34:315.

A method for obtaining a suitable nucleic acid sequence is PCR. In this method, cDNA is prepared from poly(A)+RNA or total RNA using the enzyme reverse transcriptase. Two primers, typically complementary to two separate regions of cDNA (oligonucleotides) encoding a truncated adalimumab and a polymerase such as Taq polymerase are added to the cDNA. The polymerase amplifies the cDNA region between the two primers.

An alternative to obtaining a nucleic acid sequence is screening an appropriate cDNA library (i.e., a library prepared from one or more tissue source believed to express the protein of interest) or a genomic library (a library prepared from total genomic DNA). The source of the cDNA library is typically a tissue from a species believed to express a desired protein in reasonable quantities. The source of the genomic library is a tissue(s) from a mammal or other species believed to contain a gene encoding a form of truncated adalimumab.

Certain embodiments of the invention herein provide nucleic acid molecules encoding biologically-active, half-life extended, and truncated forms of adalimumab. Accordingly, the nucleic acid molecule contains a nucleic acid sequence encoding a truncated form of a biologically active adalimumab and a nucleic acid sequence encoding an amino acid sequence, which forms and/or adopts either entirely or in part, a random coil conformation domain (RCCD), and which confers the desired half-life extension under physiological conditions. Preferably, the nucleic acid molecule is in a vector. The truncated forms are antibody fragments (e.g., Fabs), or engineered antibody fragments (e.g., scFv, diabodies, etc.), or other binding molecules (i.e., scaffolds) is described herein.

Furthermore, transfection of cells with the nucleic acid molecule or vectors is described herein. The nucleic acid molecules are fused to suitable expression control sequences known in the art to ensure proper transcription and translation of the polypeptide as well as signal sequences to ensure cellular secretion or targeting to organelles. Such vectors contain additional genes such as marker genes that allow for the selection of said vector in a suitable host cell and under suitable conditions.

Preferably, the nucleic acid molecule provided by certain embodiments of the invention herein is in a recombinant vector in which a nucleic acid molecule encoding the herein described biologically-active, half-life extended, truncated adalimumab protein is operatively linked to expression control sequences allowing expression in prokaryotic or eukaryotic cells. Expression of the nucleic acid molecule is accomplished by transcription of the nucleic acid molecule into a translatable mRNA. Regulatory elements are responsible for expression in prokaryotic host cells, e.g., the lambda PL, lac, trp, tac, tet, or T7 promoter in E. coli. Regulatory elements for expression in eukaryotic cells, preferably mammalian cells or yeast, are well known to those of ordinary skill in the art. Regulatory elements contain regulatory sequences that initiate transcription and optionally poly-A signals for termination of transcription and stabilization of the transcript. Additional regulatory elements contain transcriptional as well as translational enhancers, and/or naturally-associated or heterologous promoter regions. Examples for regulatory elements permitting expression in eukaryotic host cells are the AOX1 or GAL1 promoter in yeast or the CMV, SV40, RSV promoter (Rous sarcoma virus), CMV enhancer, SV40 enhancer or a globin intron in mammalian and other animal cells. Apart from elements that are responsible for the initiation of transcription, such regulatory elements also contain transcription termination signals, such as the SV40-poly-A site or the tk-poly-A site, downstream of the coding region Veronese (2001) Biomaterials 22 (5), 405-417.

Methods that are well known to those skilled in the art are used to construct recombinant vectors, for example, techniques described in Sambrook et al, supra. and Ausubel et al supra. Suitable expression vectors are well-known in the art, such as Okayama-Berg cDNA expression vector pcDV1 (Pharmacia), pCDM8, pRc/CMV, pcDNA1, pcDNA3, pPICZalpha A (Invitrogen), and pSPORT1 (GIBCO BRL). Depending on the expression system used, leader sequences to direct the polypeptide to a cellular compartment or to secrete the polypeptide into the culture medium are added to the coding sequence of the nucleic acid molecule provided by certain embodiments of the invention herein.

Compositions herein are in solid or liquid form such as a powder, a tablet, a solution, an aerosol, a nanoparticle, or attached to a nanoparticle. Furthermore, certain embodiments of the invention herein contain additional biologically active agents, depending on the intended use of the pharmaceutical composition.

Administration of the pharmaceutical compositions herein is performed in different ways, e.g., by parenteral, subcutaneous, intraperitoneal, topical, intra-bronchial, intra-pulmonary and intra-nasal administration and, if desired for local treatment, intra-lesional administration. Parenteral administrations include intra-peritoneal, intra-muscular, intra-dermal, subcutaneous intra-venous or intra-arterial administration. The compositions herein are administered directly to the target site by biolistic delivery to an external or an internal target site, such as a specifically effected organ.

Examples of suitable pharmaceutical carriers, excipients and/or diluents are well known in the art and include phosphate buffered saline solutions, water, emulsions, such as oil/water emulsions, various types of wetting agents, and sterile solutions, etc. Compositions containing such carriers are formulated by methods well known to one on ordinary skill in the art. Suitable carriers are well known in the art and contain material which, when combined with the biologically active protein of certain embodiments of the invention herein, retains the biological activity of the biologically active protein (Remington's Pharmaceutical Sciences, 22^{nd} Ed.; Gennaro, Mack Publishing, Easton, Pa. (2012); Remington's Pharmaceutical Sciences. 16th edition, Osol, A. Ed. 1980). Preparations for parenteral administration include sterile aqueous or non-aqueous solutions, suspensions, and emulsions. The buffers, solvents and/or excipients as employed in context of the pharmaceutical composition are preferably “physiological”. Examples of non-aqueous solvents are propylene glycol, PEG, vegetable oils such as olive oil, and injectable organic esters such as ethyl oleate. Aqueous carriers include water, alcoholic/aqueous solutions, emulsions, or suspensions including saline and buffered media. Parenteral vehicles include sodium chloride solution, Ringer's dextrose, dextrose, and sodium chloride, lactated Ringer's, or fixed oils. Intravenous vehicles include fluid and nutrient replenishes, electrolyte replenishers such as those based on Ringer's dextrose, and the like. Preservatives and other additives are also present, for example, anti-microbials, anti-oxidants, chelating agents, and inert gases. In addition, the pharmaceutical compositions of certain embodiments of the invention herein contain proteinaceous carriers, e.g., serum albumin or immunoglobulin, preferably of human origin.

These pharmaceutical compositions are administered to the subject at a suitable dose. The dosage regimen will be determined by the attending physician and clinical factors. As is well known in the medical arts, dosages for any one patient depend on many factors, including the patient's size, body surface area, age, the particular compound to be administered, sex, time and route of administration, general health, and other drugs being administered concurrently. Pharmaceutically active matter are present in amounts between 1 μg and 20 mg/kg body weight per dose, e.g. between 0.1 mg to 10 mg/kg body weight, e.g. between 0.5 mg to 5 mg/kg body weight. If the regimen is a continuous infusion, it should also be in the range of 1 μg to 10 mg per kilogram of body weight per minute. A preferred therapeutic dose achieves steady-state blood levels for the biologically-active fusion molecules provided by herein and is commensurate with the designed terminal half-life of the molecules. Yet, doses below or above the indicated exemplary ranges also are envisioned, especially considering the aforementioned factors.

Furthermore, certain embodiments of the pharmaceutical composition of the methods and compositions herein provide further biologically active agents, depending on the intended use of the pharmaceutical composition. These further biologically active agents are antibodies, antibody fragments, hormones, growth factors, enzymes, binding molecules, cytokines, chemokines, nucleic acid molecules, or drugs.

Methods herein of preventing and/or treating acute and chronic inflammation and autoimmune diseases by administering a prophylactic and/or therapeutic formulation contain a recombinant soluble human Fab′ or (Fab′)₂ of adalimumab, which have been modified either by genetic fusion or by chemical conjugation to a linear RCCD polypeptide containing natural amino acids or a combination of natural and unnatural amino acids, the polypeptide having a specific length “n”.

Fab_{adal} is a targeting agent conjugated to a polypeptide containing natural or a combination of natural and unnatural amino acids, the polypeptide having a specific length “n”, also incorporating anti-inflammatory drugs such as methotrexate to treat arthritis and other inflammatory diseases. A variety of steroidal and non-steroidal drugs, disease modifying drugs, other anti-inflammatory compounds, and immunotoxins, are conjugated to Fab_{adal} either by genetic fusion or by chemical conjugation. Fab_{adal} conjugated to a polypeptide containing proline, alanine, and/or serine accumulates at the inflamed site or in diseased cells, where the drug is released for maximum therapeutic effect.

Compositions provided herein are useful to extend the half-life of a biopharmaceutical protein drug in comparison to the protein no similarly modified. The drug circulates longer than the unmodified or PEG-modified protein in the body to treat the disease, and is stealthy to avoid rejection by the body because of immune reactions.

Present biopharmaceutical drugs for treating arthritis and other inflammatory diseases are not suitably modified for optimal half-life and fail to mask immune reactions. Embodiments of the methods and compositions herein provide masking of the immuno-suppressive nature of the biopharmaceutical drug and increase its half-life in the body. Consequently, it is not rejected by the body, does not result in immune reactions, and is administered in doses having lower quantities or in a therapeutic regimen at a lower frequency.

### Description of the Process

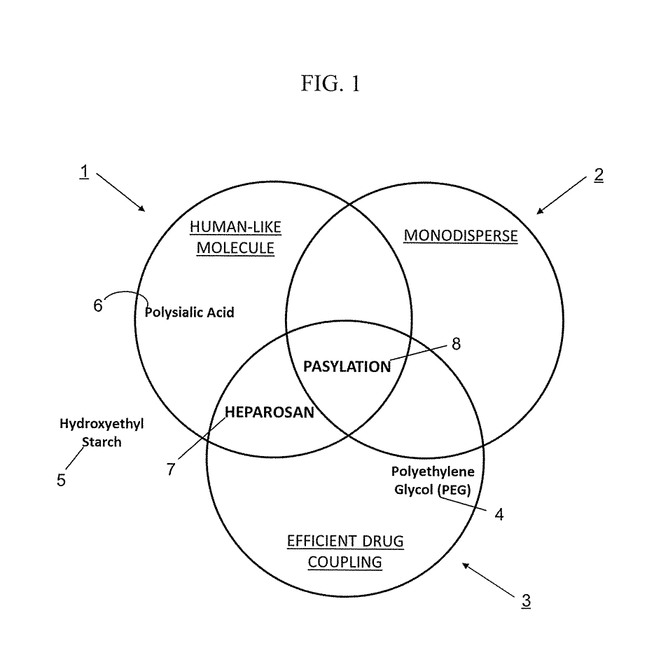

FIG. 1 is a diagram of the problem and the optimal solution provided herein to half-life extension and the effective delivery of biopharmaceutical drugs. An ideal solution is achieved when characteristics of a human-like molecule, 1, monodispersity, 2, and efficient drug coupling methods, 3, (either by genetic fusion or by chemical conjugation techniques) are combined. Prior art techniques for increasing the half-life of molecules show use of PEG, 4; hydroxyethyl starch, 5; and/or polysialic acid, 6. As noted in FIG. 1, each of the prior art techniques has characteristics that preclude them from providing the optimal half-life extension solution. PEG, 4, is a widespread half-life extension technology for biological molecules. The European Medicines Agency released warnings regarding long-term administration of drugs containing PEG, 4, because of increasing evidence of cellular vacuolation in various organs and in renal tubular cells (EMEA (2012), supra). PASylation®, 8, is a newer technology that has advanced the half-life extension/drug delivery frontier. Methods and compositions herein combine the three desired characteristics of a human-line molecule 1, monodispersity 2, and efficient drug coupling methods 3 in an optimal manner. PASylation®, 8, is a suitable modification of Fab_{adal} molecules that optimally combines these characteristics in a manner that circumvents the performance issues of the prior art methods.

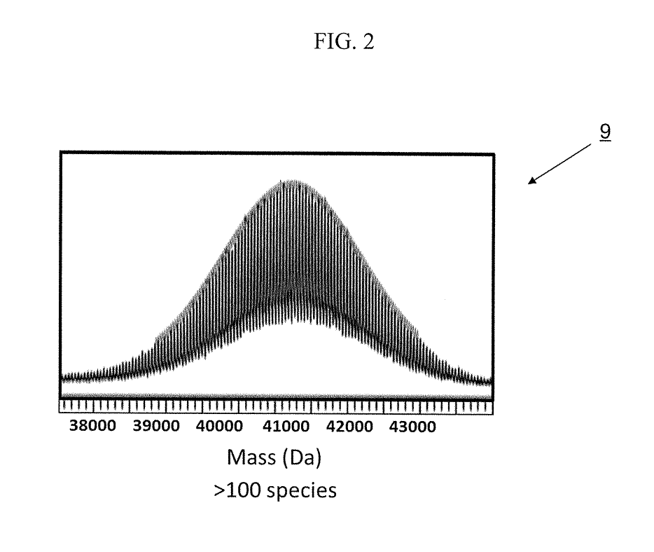

FIG. 2 illustrates the highly polydisperse nature of products of the currently available technology using PEG, 4, residues for increasing the half-lives of biopharmaceutical drugs. The highly polydisperse nature of PEG, 4, as conjugated to a drug, tends to mask the reactive site, which results in a dramatic reduction in effectiveness of the drug.

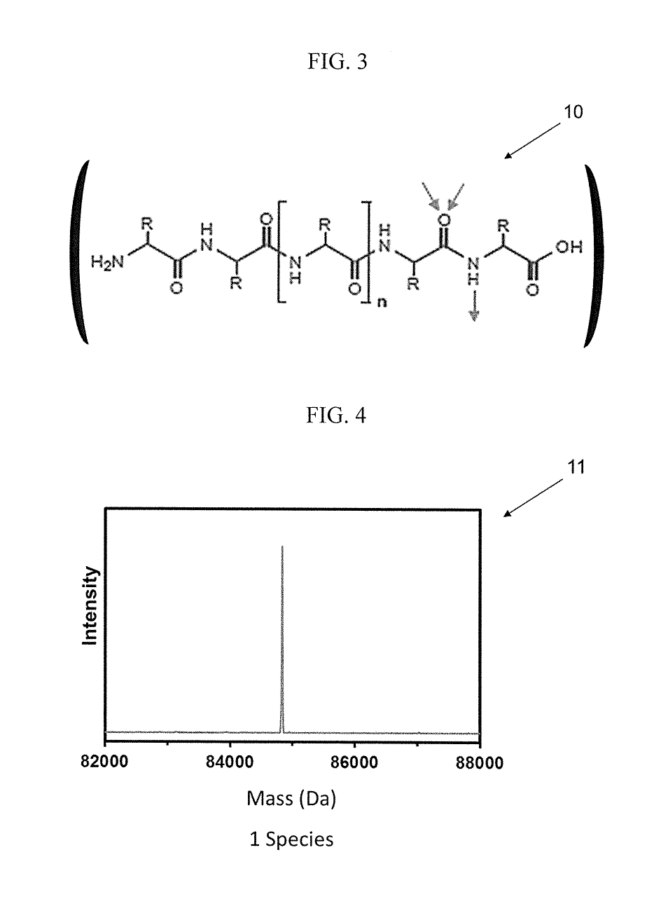

FIG. 3 is an illustration of the basis for PASylation® technology, 8, and depicts the structure and sequence of a PAS polypeptide, 10, containing natural amino acids proline, alanine, and/or serine (PAS), the polypeptide having specific length “n”. The length of each polypeptide 10 is in the range of about 200 amino acid residues to about 1,200 amino acid residues, about 20 amino acid residues to about 600 amino acid residues, about 50 amino acid residues to about 800 amino acid residues, about 100 amino acid residues to about 1,000 amino acid residues, about 150 amino acid residues to about 1,100 amino acid residues, and about 300 amino acid residues to about 1,500 amino acid residues. Alternatively, the PAS polypeptide, 10, is greater than 1,200 amino acid residues long. The length chosen by a user depends on the desired half-life extension. The immune system of the body does not recognize the PAS polypeptide, 10, as being foreign and hence does not elicit an immune-response, unlike observations with PEG, 4, because the PAS polypeptide, 10, contains natural amino acids. Alternatively, the PAS polypeptide, 10, is combined with unnatural amino acids, if required for a particular function. The PAS polypeptide, 10, is genetically fused at the encoding nucleic acid level to the gene encoding the biopharmaceutical drug for simultaneous expression of a resulting fusion protein, or it can be chemically conjugated, unlike the other technologies depicted in FIG. 1.

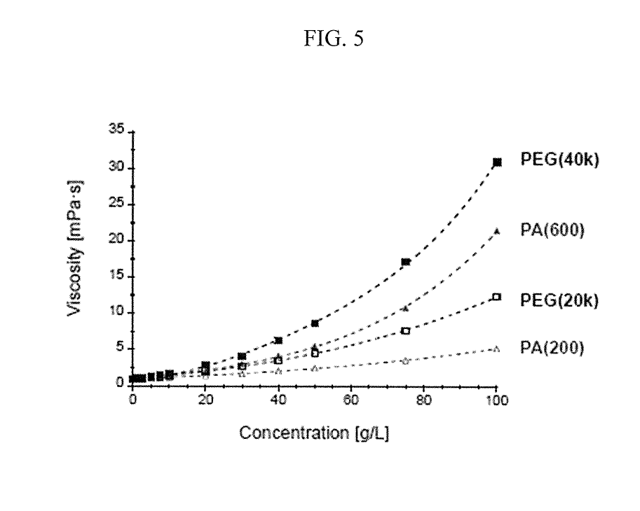

FIG. 4 is a graph of mass spectroscopy data indicating that the PAS polypeptide, 10, is a single-species homogeneity and is monodisperse.

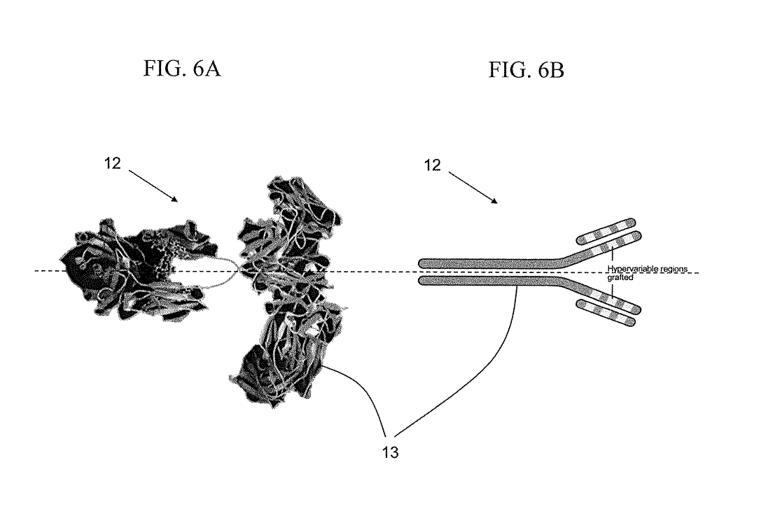

FIG. 5 is a graph that compares the viscosities of polymers having various lengths of amino acid residues exemplified by the repeating structure of FIG. 3, and PEG polymers in the preferred molecular weight range. Viscosity was measured with a μVISC™ microviscometer with VROC® chip in Phosphate Buffered Saline. In FIG. 5, the PAS polypeptide, 10, and the PEG are unconjugated, i.e., independent of proteins, thereby depicting the inherent baseline viscosities of each potential adduct. Viscosities of PASylated or PEGylated drugs are strongly influenced by fusion and conjugation partner(s). It was observed that the hydrodynamic volume of the PA(200) polypeptide chain corresponds to a PEG polymer of molecular weight 20 kDa (PEG(20k)), while the hydrodynamic volume of the PA(600) polypeptide chain roughly corresponds to a PEG molecule of molecular weight 40 kDa (PEG(40k)). For corresponding hydrodynamic volumes at higher concentrations, the PAS polypeptides were observed to have viscosities that are one-third to three-fold lower than the PEG molecules.

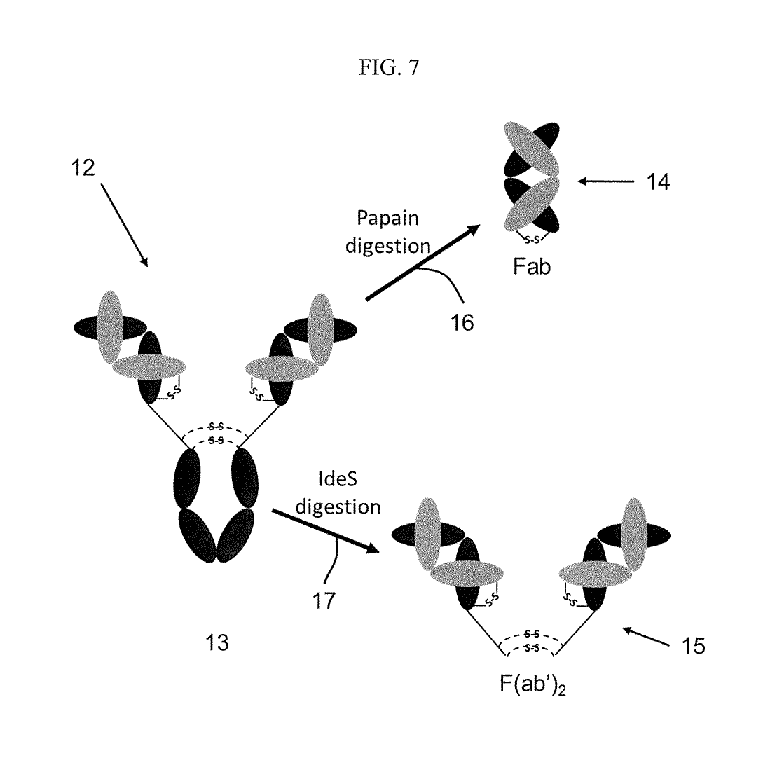

FIG. 6A is a three-dimensional ribbon model of x-ray crystallography data of a full-length adalimumab antibody.

FIG. 6B is an illustration of the structure of a full-length adalimumab antibody. The antibody consists of 1330 amino acids and has a molecular weight of approximately 148 kDa (EMA Report 2004 supra).

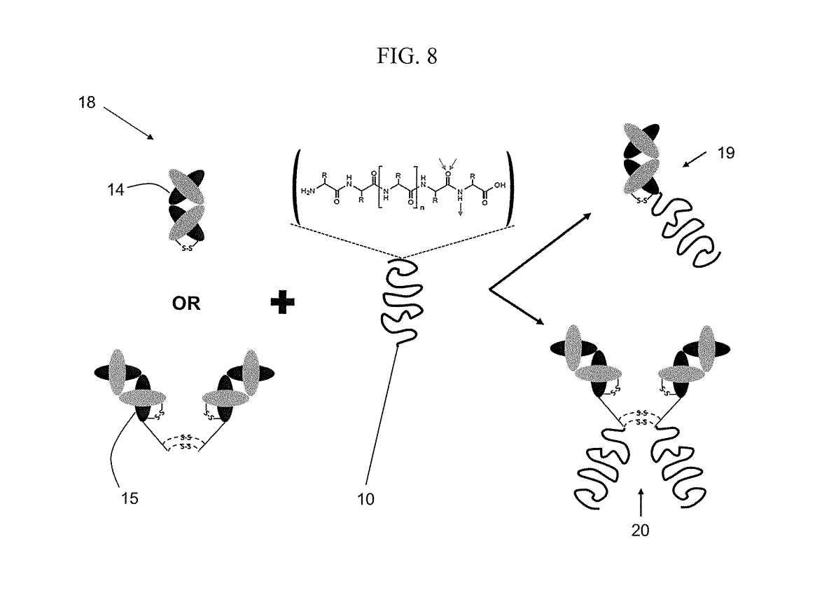

FIG. 7 is an illustration of a design for a process of engineering of two types of antibody fragments—a Fab, 14, and a F(ab′)₂, 15, from a full-length antibody, 13, one having ordinary skill in the art (Cresswell et al. (2005) Biotechnol. Appl. Biochem. 42 (2), 163; Rousseaux et al (1983) J. Immunol. Methods 64 (1-2), 141-146; Mitchel et al. (1970) J. Biol. Chem. 245 (14), 3485-3492).

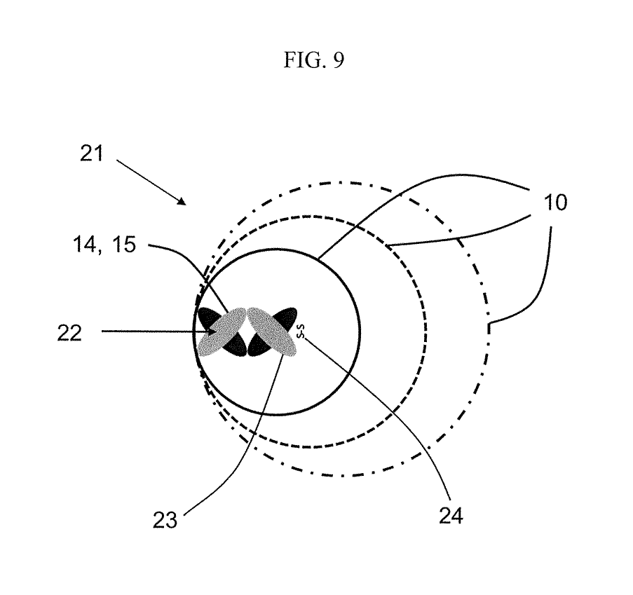

FIG. 8 is an illustration of the process through recombinant molecular biology principles of combining a Fab, 14, at least one with PAS polypeptide, 10, or variants thereof to create a biologically active pharmaceutical composition, 19, or combining a fragment of F(ab′)₂, 15, with a PAS polypeptide, 10, or variants thereof to produce a biologically active pharmaceutical composition, 20 (Skerra et al., WO2008155134A1, supra). FIG. 8 depicts conjugation—either by genetic fusion, or by chemical means—at either the N terminus and/or the C terminus of a Fab, 14, or a F(ab′)₂, 15.

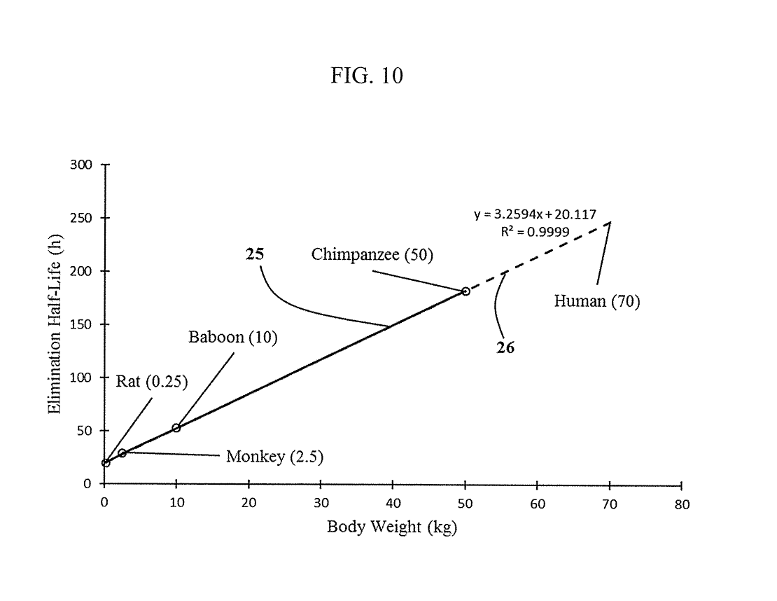

FIG. 9 is an illustration of the beneficial effects of administration of the pharmaceutical composition proteins in FIG. 8. These compositions were observed to have successful functions—the reactive site, 22, in either Fab, 14, or F(ab′)₂, 15, remains open and unhindered, while the immunogenic sites, 23, on both are masked by the picosecond to femtosecond vibrations of the PAS polypeptide, 10, and/or variants thereof, which creates the hydrodynamic cloud indicated by the dashed circles. The effective hydrodynamic volume in FIG. 9 is directly dependent on the number of amino acid residues in the sequence of the PAS polypeptide, 10, with the increase in circle diameters correlating to increasing lengths of the polypeptide. The disulfide linkage, 24, was observed to be protected by the hydrodynamic cloud created by the PAS polypeptide, 10, and variants thereof.

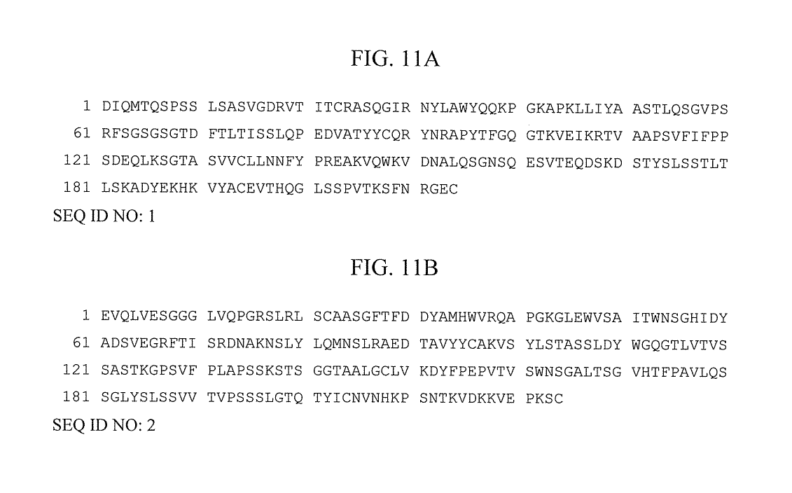

FIG. 10 is a graph of the elimination half-life of the biologically active pharmaceutical composition, 19, containing Fab, 14, linked to a PAS polypeptide, 10, or variants thereof, or the biologically active pharmaceutical composition, 20, containing F(ab′)₂, 15, linked to a PAS polypeptide, 10, or variants thereof, as a function of body weight. The volume of distribution and plasma clearance of protein pharmaceuticals over a wide molecular weight range (6,000 to 98,000 Daltons) followed well-defined, size-related physiological relations. Preclinical pharmacokinetic studies provided estimates of human disposition after interspecies scaling. (Grene-Lerouge et al. (1996) Toxicol. Appl. Pharmacol. 138, 84. 1996; Caldwell, G. W. et al. (2004) Eur. J. Drug. Metab. Pharmacokinet. April-June; 29 (2):133-43). The elimination half-life/plasma clearance data for the biologically active pharmaceutical composition, 19, containing Fab, 14, linked to a PAS polypeptide, 10, or variants thereof, or the biologically active pharmaceutical composition, 20, containing F(ab′)₂ 15 linked to a PAS polypeptide 10 or variants thereof were scaled from the values observed in rats, monkeys, baboons, and chimpanzees, 25, to predict the pharmacokinetics in humans, 26. As chimpanzees (Pan troglodytes) are the closest relative to humans and are of a similar body weight (50 kg and 70 kg, respectively) the pharmacokinetics in chimpanzees are expected to be similar to those in humans. For a 70 kg human, the elimination half-life of either the biologically active pharmaceutical composition, 19, containing Fab, 14, linked to a PAS polypeptide, 10, or variants thereof, or the biologically active pharmaceutical composition, 20, containing F(ab′)₂, 15, linked to a PAS polypeptide, 10, or variants thereof, is predicted to be approximately 250 hours. The correlation coefficient between actual data, 25, and the prediction for the half-life of the biologically active pharmaceutical composition, 19, containing Fab, 14, linked to a PAS polypeptide, 10, or variants thereof, or the biologically active pharmaceutical composition, 20, containing F(ab′)₂, 15, linked to a PAS polypeptide, 10, or variants thereof in humans, 26, as calculated by the equation in FIG. 10, is high compared to PEGylated pharmaceutical compositions.

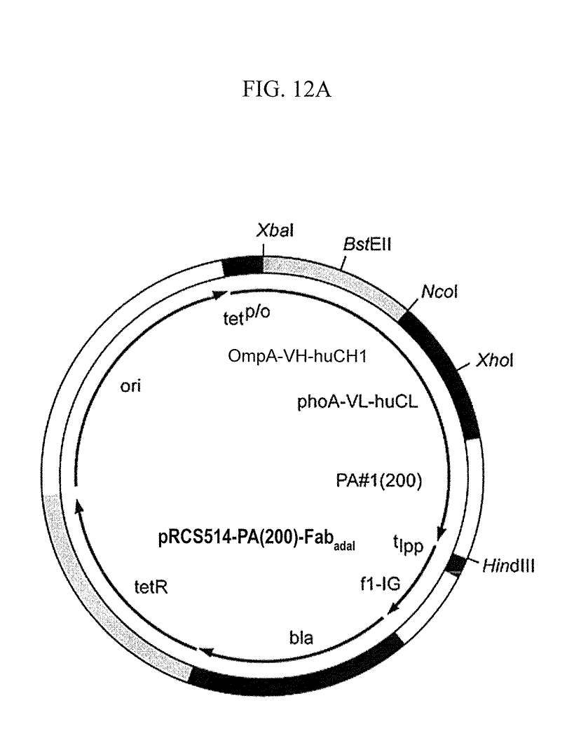

FIG. 11A-11C are the amino acid sequences of the light and heavy chains of the adalimumab antibody, respectively (DrugBank Accession No. DB00051). As shown in FIG. 11A, the LC contains 214 amino acid residues. As shown in FIG. 11B, the HC contains 224 amino acid residues. The combined amino acid sequences of both the LCs and both the HCs were used to predict the three-dimensional structure of the full protein molecule, 16, of FIG. 6A. For generation of antibody fragments from an entire antibody, certain embodiments of the methods and compositions herein provide methods to create Fab fragments as shown in FIG. 7, which are either genetically-fused to or chemically-conjugated to appropriately-sized PAS polypeptides. FIG. 11C is an exemplary amino acid sequence of a PAS polypeptide: ASPAAPAPASPAAPAPSAPA (SEQ ID NO: 3).

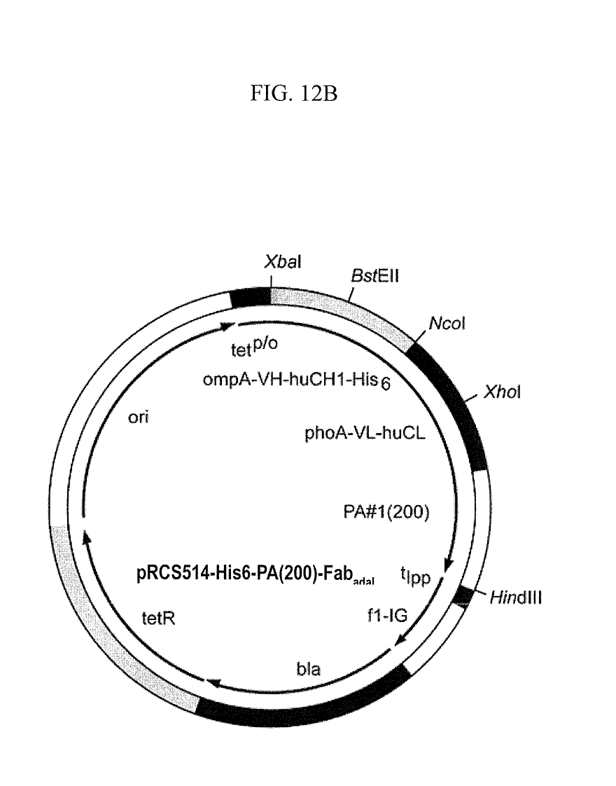

FIG. 12A and FIG. 12B are illustrations of two embodiments of the cloned construct and a plasmid map showing suitable restriction sites for genetically fusing a PAS polymer sequence to a representative fragment of adalimumab (Fab_{adal}). The structural genes for the HC and LC in plasmid pRCS514-PA(200)-Fab_{adal} are under transcriptional control of the tetracycline promoter/operator (tet^{p/o}) and the operon ends with the lipoprotein terminator (tₗₚₚ). FIG. 12A is an illustration of the HC containing the bacterial OmpA signal peptide, the variable (VH), and the first human IgG1 heavy chain constant C domain (CH). Plasmid pRCS514-His6-PA(200)-Fab_{adal}) shown in FIG. 12B, was designed so that the HC is tagged with an affinity tag, a Histidine polypeptide with six residues (e.g. OmpA-VH-huCH1-His₆), to aid in downstream chromatographic purification of the antibody fragment, Fab_{ada1}, using well-established metal-chelate affinity chromatographic techniques (Petty, K. J. (2001) Current Protocols in Protein Science. University of Texas Southwestern Medical Center, Dallas. Wiley). Alternative embodiments contain other tags known in the art, as part of the HC sequence, to simplify the purification process. The LC contains the bacterial PhoA signal peptide, the variable (VL) and human LC constant (CL) domain, and the PA polypeptide with 200 amino acid residues around the C-terminus of the immunoglobulin LC of Fab_{adal}. The plasmid backbone of pRCS514-PA(200)-Fab_{adal} outside the expression cassette flanked by the XbaI and HindIII restriction sites is identical with that of a generic cloning and expression vector (Skerra, A. (1994) Gene 151:131-135). Singular restriction sites are indicated.

The expression vectors for PAS400-, PAS600-, PAS800-, PAS1,000-, or PAS1,200-antibody or antibody fragment contain, respectively, the PAS#1 polymer with 400-, 600-, 800-, 1,000- or 1,200 amino acid residues or more, encoded by a corresponding gene cassette instead of PAS(#1)200, and are otherwise identical. An exemplary amino acid sequence of PAS#1 is ASPAAPAPASPAAPAPSAPA (SEQ ID NO: 3).

FIG. 11A provides an exemplary amino acid
sequence of the LC:
(SEQ ID NO: 1)
DIQMTQSPSSLSASVGDRVTITCRASQGIRNYLAWYQQKPGKAPK
LLIYAASTLQSGVPSRFSGSGSGTDFTLTISSLQPEDVATYYCQR
YNRAPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.
FIG. 11B provides an exemplary amino acid
sequence of the HC:
(SEQ ID NO: 2)
EVQLVESGGGLVQPGRSLRLSCAASGFTFDDYAMHWVRQAPGKGL
EWVSAITWNSGHIDYADSVEGRFTISRDNAKNSLYLQMNSLRAED
TAVYYCAKVSYLSTASSLDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSC.

In additional embodiments, the amino acid sequences contain conservative amino acid mutations, which are mutations that change an amino acid to a different amino acid with similar biochemical properties, for example, the properties of charge, hydrophobicity, and size. For example, leucine and isoleucine are aliphatic, branched, and hydrophobic. Similarly, aspartic acid and glutamic acid are both small, negatively charged amino acid residues. Conservative mutations in proteins often have a smaller effect on function than non-conservative mutations and are accordingly less likely to disrupt protein structure and/or function.

Amino acids are classified into groups on the basis of their structure and the general chemical characteristics of their R groups:

Aliphatic—glycine (G), alanine (A), valine (V), leucine (L), isoleucine (I)

Hydroxyl or sulfur-containing—serine (S), cysteine (C), threonine (T), methionine (M)

Cyclic—proline (P)

Aromatic—phenylalanine (F), tyrosine (Y), tryptophan (W)

Basic—histidine (H), lysine (K), arginine (R)

Acidic and Amide—aspartate (D), glutamate (E), asparagine (N), glutamine (Q)

These groups contain amino acids used to design substitution variants envisioned as within the scope of the compositions.

Expression of a PASylated form of one of an antibody or antibody fragment is a process familiar to one of ordinary skill in the art. The genetic fusion of a PAS sequence with any one of the forms of antibodies or antibody fragments was expressed either in the cytoplasmic space of an E. coli host, or in the periplasmic space of E. coli. Alternatively, eukaryotic expression hosts (e.g. CHO) are within the scope of the methods. For periplasmic expression, a nucleic acid sequence such ‘ATG’ was added as a start codon to the N-terminus of the antibody or antibody fragment gene of interest. The start codon was followed by a signal peptide such as the OmpA periplasmic signal sequence, which was followed by two unique type ITS SapI restriction sites upstream of the antibody or antibody fragment gene sequence. A stop codon, for example, the nucleic acid sequence ‘TAA’ was added at the C-terminus of the antibody or antibody fragment gene. Using a combination of restriction enzymes and ligases, the SapI amino acid sequence was spliced out, leaving “sticky” ends so that a PAS gene sequence cassette with complimentary “sticky” ends was inserted by ligation to create the PAS-antibody or -antibody fragment gene to be inserted by known plasmid-insertion techniques into the appropriate host for expression of a PAS-modified antibody or antibody fragment.

### Methods of Making Embodiments of the Invention Herein

Certain embodiments of the compositions herein are created using knowledge of basic molecular biology techniques (Sambrook et al., supra) and/or basic chemical reactions (for example, thiol-, or alkyl-, or aldehyde chemistries).

A variety of steroidal and non-steroidal drugs, disease modifying drugs, anti-inflammatory compounds, and immunotoxins are incorporated into the Fab_{adal} modified by conjugation to an RCCD polypeptide containing natural or unnatural amino acids, the polypeptide having specific length “n”.

### Techniques of Use

Embodiments of the methods and compositions provided herein are used by medical doctors and practitioners to treat patients suffering from life-long diseases such as arthritis and other inflammatory and autoimmune diseases, and for indications such as Alzheimer's disease, cancer, and other related disorders.

### Examples (1-7). Increases in Half-Lives in Balb/C Mice of PAS Compositions

TABLE 1

| | | Half-life of | Fold |
|---|---|---|---|
| Ex. | | modified | increase in |
| no. | Protein sample | molecule | half-life |
| 1 | Unmodified antibody Fragment (Fab) | 1.3 | 1.0 |
| 2 | Fab with 1 backbone of 100 PAS residues | 2.7 | 2.0 |
| 3 | Fab with 1 backbone of 200 PAS residues | 5.2 | 3.9 |
| 4 | Fab with 1 backbone of 400 PAS residues | 14.4 | 10.7 |
| 5 | Fab with 1 backbone of 600 PAS residues | 28.2 | 21.0 |
| 6 | Fab with 2 backbones of 200 PAS residues | 37.2 | 27.8 |
| | each | | |
| 7 | Fab-ABD | 28.9 | 21.6 |

### Effect of PASylation® on the Half-Life of a Fab Payload

Table 1 contains data on the effect of PASylation® on the half-life of an unmodified antibody fragment (Fab) from mouse preclinical analysis. In Examples 1-5, a clear relation was observed between an increased number of PAS residues and an increase in the half-life of the Fab. Example 6 shown that a Fab containing two polypeptides of 200 PAS residues were observed to result in an increase in the half-life of the Fab compared to the half-life of Fab conjugated to one polypeptide of 400 amino acid residues. The two polypeptides of 200 amino acid residues each are conjugated to two different locations on the Fab having the effect of creating a larger effective molecular volume than one polypeptide of 400 PAS residues. This increased half-life functions well with antibody types of proteins because an antibody affords multiple locations for conjugation. The range of length of PAS residues and the type and nature of the Fab payload used in the Examples of Table 1 are exemplary only and are not intended to restrict the upper end of the length of the PAS polypeptides, or the type and nature of the Fab antibodies that are modified. Certain embodiments of the compositions herein provide PAS residues are added to the polypeptide to extend the length well beyond 1,200 amino acid residues, and the length is determined by particular clinical needs of each Fab payload.

### Interspecies Allometric Scaling

Interspecies allometric scaling illustrated in FIG. 10 was used to predict the half-life of a PAS-Fab_{adal} in humans based on data from Table 1. The half-life is projected to be in about 250 hours for a human of 70 kg body weight. This half-life is a substantial increase over unmodified Fab, and results in improved treatments for arthritis and related autoimmune diseases. Concomitant improvements in patient compliance, cost of treatment, and clinical burden also result from extended half-life. A current, established treatment for rheumatoid arthritis (RA) is adalimumab (Humira®; AbbVie, Inc. Chicago, Ill.). Since Humira® is a full-length antibody, it does not necessarily require half-life modification. Humira® costs approximately $3,100 per month, and had global sales in 2015 of over $12 billion partly due to its complicated manufacturing process based on mammalian cell technology (U. S. Pharmaceutical Statistics (February 2014), www.drugs.com/stats/top100/2012/sales; Abbot Press Release (March 2012), www.abbott.mediaroom.com/2012-03-21). Developers have launched biosimilar versions, at prices around $200 per vial (Reuters (Dec. 9, 2014), www.reuters.com, USKBN0JN0X820141209).

Examples 3-6 in Table 1 indicate the ability to tune the half-life of a drug on an a priori basis and in a precise manner. One polypeptide of 200 PAS residues was observed to produce a 4-fold increase in half-life of Fab (Example 3), and two polypeptides of 200 PAS residues were observed to produce a 28-fold increase in the half-life of the same Fab (Example 6). One polypeptide of 400 PAS residues results in an 11-fold increase in the half-life (Example 4), and one polypeptide of 600 PAS residues was observed to result in a 21-fold increase in the half-life of the unmodified Fab (Example 5). The difference between the half-life of Fab in Example 6 and the half-life of Fab in Example 3 is 7-fold. Therefore, on the basis of the same relationship, two polypeptides of 400 PAS residues, or two polypeptides of 600 PAS residues used instead of their single polypeptide counterparts, are predicted to double polypeptide constructs to result in a 77-fold (11×7) and a 147-fold (21×7) increase in the respective half-life of each.

Extrapolating these results from mouse data to humans, results in estimates of a half-life of over 400 hours and 800 hours, respectively, for the double PAS polypeptide-400 or the double PAS polypeptide-600 amino acid residue constructs, using a scaling factor of four from mice data to human data (Caldwell et al. supra). Embodiments of the composition and methods of treatment herein reduce the frequency of daily injections for treatments compared to treatments currently in use, to once per week, once per two weeks, or once per month, thereby altering dynamics of treatment and improving compliance for patients suffering from RA and chronic inflammation-related diseases. Methods and compositions herein provide a potential long-term (greater than 3-5 years) benefit to patients, compared to a 6-12 month time frame typical of other treatments. Additionally, clinical burden and cost of treatment are reduced which positively affects the burden of increasing healthcare costs.

Methods and compositions herein provide a half-life modification of a full-length antibody, 13, and/or variants thereof, Fab, 14, and/or F(ab′)₂, 15, by PASylation®. The process is contrasted herein with PEGylation, which has technical and performance issues relating to high viscosities, rendering its preparation and formulation for injection a difficult task to accomplish. The PAS polypeptide has a lower viscosity than PEG. Furthermore, there are no cost of goods issues with the process of PASylation® because the preferred mode of expression is simultaneous with the product as a genetically-fused product. Alternatively, PASylation® is performed by a post-production, chemically-conjugated process, which is the only method by which PEGylation is performed.

1. A composition for preventing or treating a subject for at least one of an inflammation, an autoimmune disease, a neurological disease, and a cancer, the composition comprising:
a full-length antibody or a functional antibody fragment that is an anti-human TNFα antibody; and
an adduct covalently linked to the full-length antibody or the functional antibody fragment that increases half-life of the composition in the subject, and the composition having decreased immunogenicity than the full-length antibody or the functional antibody fragment alone, or than a corresponding PEGylated form of the full-length antibody or the functional antibody fragment.

2. The composition according to claim 1, wherein the full-length antibody or the functional antibody fragment comprises at least one of the following characteristics: the full-length antibody or the functional antibody fragment is selected from the antibody classes of proteins consisting of: IgG, IgM, IgA, IgD, and IgE, for example is from the IgG class; the functional antibody fragment is a Fab or a F(ab′)₂: the full-length antibody or the functional antibody fragment is adalimumab; the full-length antibody or the functional antibody fragment is human or humanized; the Fab or the F(ab′)₂ is a recombinant mutagenized protein; and/or the Fab or the F(ab′)₂ is a proteolytic product of a digest of the full-length antibody.

3-10. (canceled)

11. The composition according to claim 1, wherein the Fab or the F(ab′)₂ is encoded by a nucleic acid obtained by at least one technique selected from the group consisting of: chemical synthesis, cDNA, genomic library screening, expression library screening, and polymerase chain reaction (PCR).

12. (canceled)

13. The composition according to claim 1, wherein the adduct comprises at least of the following: a polypeptide containing proline and alanine; a polypeptide containing proline, alanine, and serine (PAS polypeptide); and/or naturally occurring sugars selected from at least one of glucuronic acid, N-acetylglucosamine, and heparosan.

14-18. (canceled)

19. The composition according to claim 1, wherein the adduct comprises a linear polypeptide containing natural and/or unnatural amino acid residues or comprises a nonlinear polypeptide.

20. (canceled)

21. The composition according to claim 1, wherein the adduct increases in vivo half-life of the full-length antibody or the functional antibody fragment by a factor of at least about 10-fold or by a factor of at least about 300-fold, for example, the half-life is at least about 25 hours, at least about 125 hours, or at least about 275 hours.

22. (canceled)

23. The composition according to claim 1, wherein the PAS polypeptide forms a monodisperse mixture.

24. The composition according to claim 1, wherein the composition structure is further characterized by at least one of: the adduct is covalently linked at the C terminus of the full-length antibody or the functional antibody fragment or the N terminus of the full-length antibody or the functional antibody fragment; the adduct is a plurality of adducts, and a first adduct is covalently linked at the N terminus and a second adduct is covalently linked at the C terminus of the full-length antibody or the functional antibody fragment; the adduct is covalently linked to the full-length antibody or the functional antibody fragment at a position internal to the N terminus or the C terminus; the adduct is a plurality of adducts, and each of the plurality is covalently linked to one of a plurality of positions on the full-length antibody or the functional antibody fragment; and, the covalent linkage comprises of two adducts, each having a length of at least about 200 amino acid residues.

25-27. (canceled)

28. The composition according to claim 1, wherein the adduct further comprises at least one drug selected from the group consisting of: an anti-inflammatory drug, for example, methotrexate, a steroidal drug, a non-steroidal drug, and an immunotoxin.

29. (canceled)

30. The composition according to claim 1, having characteristics selected from the group of: the adduct is located at or in close proximity of an immunogenic site of the full-length antibody or the functional antibody fragment and masks immunogenicity; the composition accumulates at an inflamed site or in diseased cells to treat the subject; the adduct forms a random coil conformation domain (RCCD); and/or, the composition is biodegradable in vivo in the subject.

31. The composition according to claim 1, wherein the adduct is at least about 200 amino acid residues or is at least about 1200 amino acid residues.

32-33. (canceled)

34. The composition according to claim 1, wherein the half-life in vivo is at least about 25 hours, at least about 75 hours, at least about 125 hours, at least about 175 hours, at least about 225 hours, or at least about 275 hours.

35. The composition according to claim 1, wherein the composition further comprises an affinity tag for chromatographic purification.

36-37. (canceled)

38. A method of preventing or treating a subject for at least one of an inflammation, an autoimmune disease, a neurological disease, and a cancer, the method comprising:
engineering a composition comprising a full-length antibody or a functional antibody fragment that is a Fab or a F(ab′)₂ covalently bound to an adduct, the composition increasing the half-life of the composition in the subject, and the composition containing the adduct is either the same as, or less immunogenic than that of the full-length antibody or the functional antibody fragment which is PEGylated; and
administering the composition to the subject.

39. The method according to claim 38, the method further comprising prior to administering, formulating the composition in a form that is effective for a prophylactic use or a therapeutic use.

40. (canceled)

41. The method according to claim 38, wherein the engineering step comprises at least one of the following steps: covalently binding an adalimumab to the adduct; genetically or chemically conjugating the adduct to the full-length antibody or the functional antibody fragment; mutagenizing a gene encoding the antibody or fragment and expressing the gene; conjugating a PAS polypeptide or naturally occurring sugar molecules comprising heparosan, to the full-length antibody or the functional antibody fragment; expressing the genetically conjugated antibody or fragment in prokaryotic or eukaryotic cells.

42-43. (canceled)

44. The method according to claim 38, the method further comprises prior to administering, conjugating a PAS polypeptide or naturally occurring sugar molecules comprising heparosan, to the full-length antibody or the functional antibody fragment, and increasing the half-life of the full-length antibody or the functional antibody fragment.

45-48. (canceled)

49. The method according to claim 38, wherein prior to administering, the method further comprises digesting of the full-length antibody to form the Fab or the F(ab′)₂.

50. A composition for preventing or treating a subject for at least one of an inflammation, an autoimmune disease, a neurological disease, and a cancer, the composition comprising:
a functional antibody fragment of an anti-human TNFα antibody; and
a plurality of adducts, each covalently linked to the functional antibody fragment that increases half-life of the composition in the subject, the composition having the same as, or decreased immunogenicity compared to the corresponding functional antibody fragment absent the adducts, or than the corresponding functional antibody fragment, which is PEGylated.

51. The composition according to claim 50, wherein a first adduct is linked to the C terminus of the functional antibody fragment, and a second adduct is linked to the N terminus of the functional antibody fragment.

52. The composition according to claim 50, wherein the functional antibody fragment is an adalimumab.

53. The composition according to claim 52, wherein the light chain of the adalimumab comprises an amino acid sequence of SEQ ID NO: 1 or SEQ ID NO: 2.

54. (canceled)

55. The composition according to claim 50, wherein the plurality of adducts is a PAS polypeptide, for example, the PAS polypeptide comprises about 200 amino acid residues.

56. (canceled)
